(19) United States
(12) Patent Application Publication
(10) Pub. No.: US 2024/0051910 A1
(43) Pub. Date: Feb. 15, 2024

(54) METHOD FOR PRODUCING GLYCOLIC ACID

(71) Applicant: RHODIA OPERATIONS, Lyon (FR)

(72) Inventors: Fangzheng Su, Shanghai (CN); Isabelle Storet, Lyon (FR); Stephane Streiff, Lyon (FR)

(73) Assignee: RHODIA OPERATIONS, Lyon (FR)

(21) Appl. No.: 18/259,002

(22) PCT Filed: Dec. 22, 2021
(86) PCT No.: PCT/CN2021/140281

§ 371 Date: Jun. 22, 2023

(30) Foreign Application Priority Data
Dec. 22, 2020 (WO) PCT/CN2020/138279

(51) Int. Cl. C07C 51/367; C07C 59/06; B01J 23/44; B01J 25/02; B01J 21/18

(52) U.S. Cl. CPC C07C 51/367; C07C 59/06; B01J 23/44; B01J 25/02; B01J 21/18

(57) ABSTRACT

A method for producing glycolic acid includes selectively hydrogenating glyoxylic acid using a catalyst comprising at least one transition metal element, the glycolic acid produced, which has a bio-based carbon content of above 50%. A method for preparing a composition selected from the group consisting of cosmetics, home care products, personal care products, textiles, food, beverages, drugs, fragrances, inks, or paints includes adding the glycolic acid to the composition.

This application claims priority filed on 22 Dec. 2020 in INTERNATIONAL PROCEDURE with Nr CN2020/138279, the whole content of this application being incorporated herein by reference for all purposes.

### TECHNICAL FIELD

The present invention relates to a method for producing glycolic acid. In particular, the glycolic acid is produced by selectively hydrogenating glyoxylic acid using a catalyst comprising at least one transition metal element. The present invention also relates to the glycolic acid produced by the present invention.

### BACKGROUND ART

Glycolic acid (HOCH₂COOH; IUPAC name 2-Hydroxyethanoic acid) is the smallest α-hydroxy acid. This colorless, odorless, and hygroscopic crystalline solid is highly soluble in water.

Glycolic acid is used in the textile industry as a dyeing and tanning agent, in food processing as a flavoring agent and as a preservative, and in the pharmaceutical industry as a skin care agent. It is also used in adhesives and plastics. Glycolic acid is often included in emulsion polymers, solvents and additives for ink and paint in order to improve flow properties and impart gloss. It is used in surface treatment products that increase the coefficient of friction on tile flooring. Due to its capability to penetrate skin, glycolic acid finds applications in skin care products, most often as a chemical peel.

Glycolic acid is a useful intermediate for organic synthesis, in a range of reactions including oxidation-reduction, esterification and long chain polymerization.

Glycolic acid can be synthesized in various ways.

The predominant approaches use a catalyzed reaction of formaldehyde with synthesis gas (carbonylation of formaldehyde), for its low cost (U.S. Pat. No. 2,152,852A).

US20190248724A1 discloses a process to produce glycolic acid by reacting formaldehyde with carbon monoxide and water in a carbonylation reactor in the presence of a sulfur catalyst.

US20110166383A1 discloses a process for the production of glycolic acid comprising contacting carbon monoxide and formaldehyde with a catalyst comprising an acidic polyoxometalate compound encapsulated within the pores of a zeolite.

Glycolic acid is also prepared by the reaction of chloroacetic acid with sodium hydroxide followed by re-acidification.

Other methods, not noticeably in use, include hydrogenation of oxalic acid, and hydrolysis of the cyanohydrin derived from formaldehyde (Karlheinz Miltenberger, “Hydroxycarboxylic Acids, Aliphatic” in Ullmann's Encyclopedia of Industrial Chemistry, Wiley-VCH, Weinheim, 2005).

WO2017134139A1 discloses a method of preparing glycolic acid by hydrogenation of aqueous oxalic acid in the presence of a hydrogenation metal catalyst and hydrogen.

Glycolic acid can be isolated from natural sources, such as sugarcane, sugar beets, pineapple, cantaloupe and unripe grapes.

There remains a need in the art to provide new methods to produce glycolic acid with higher conversion rate and selectivity in an economically feasible way at industrial scale.

### BRIEF DESCRIPTION OF THE INVENTION

The inventors of the present invention unexpectedly discovered that commercial catalysts were suitable to realize a selective hydrogenation of glyoxylic acid to glycolic acid under mild reaction conditions, with high selectivity for glycolic acid.

The present invention provides hereafter a new method for producing glycolic acid by selectively hydrogenation of glyoxylic acid using a catalyst comprising at least one transition metal element, with higher conversion rate and selectivity, at industrial scale, and with a reasonable cost when compared to the other available methods.

One subject matter of the invention is a method for producing glycolic acid comprising selectively hydrogenating glyoxylic acid using a catalyst comprising at least one transition metal element.

Another subject matter of the invention is a method for producing glycolic acid comprising the following steps:
(a) providing an aqueous glyoxylic acid;
(b) subjecting the aqueous glyoxylic acid to selective hydrogenation in the presence of hydrogen and a catalyst comprising at least one transition metal element, thereby obtaining glycolic acid; and
(c) collecting the obtained glycolic acid.

Another subject matter of the invention is the glycolic acid produced by the method of the present invention.

Another subject matter of the invention is a glycolic acid with a bio-based carbon content of above 50%.

Another subject matter of the invention is the use of the glycolic acid of the present invention in cosmetics, home care products, personal care products, textiles, food, beverages, drugs, fragrances, inks or paints.

### DETAILED DESCRIPTION OF THE INVENTION

For convenience, before further description of the present disclosure, certain terms employed in the specification, and examples are collected here and understood as by a person of skill in the art. The terms used herein have the meanings recognized and known to those of skill in the art, however, for convenience and completeness, particular terms and their meanings are set forth below.

The articles “a”, “an” and “the” are used to refer to one or to more than one (i.e., to at least one) of the grammatical object of the article.

The term “and/or” includes the meanings “and”, “or” and also all the other possible combinations of the elements connected to this term.

Throughout the description, including the claims, the term “comprising one” should be understood as being synonymous with the term “comprising at least one”, unless otherwise specified, and “between” should be understood as being inclusive of the limits. It is specified that, in the continuation of the description, unless otherwise indicated, the values at the limits are included in the ranges of values which are given.

The expression “comprise” should be understood as including equally “consist of” or “consist substantively of”.

It should be noted that in specifying any numerical range, such as a range of concentration, conversion rate or selectivity, any particular upper limit can be associated with any particular lower limit.

If not specified otherwise, a percentage content is on weight basis.

In the present invention, the expressions “bio-based material”, “bio-sourced material” or “natural material” designate a product that is composed, in whole or in significant part, of biological products or renewable agricultural materials (including plant, animal, and marine materials) or forestry materials.

In the present invention, the expression “bio-based carbon” refers to carbon of renewable origin like agricultural, plant, animal, fungi, microorganisms, marine, or forestry materials living in a natural environment in equilibrium with the atmosphere. The bio-based carbon content is typically evaluated by the means of the carbon-14 dating (also referred to as carbon dating or radiocarbon dating). Furthermore, in the present invention, the “bio-based carbon content” refers to the molar ratio of bio-based carbon to the total carbon of the compound or the product. The bio-based carbon content can preferably be measured by a method consisting in measuring decay process of 14C (carbon-14), in disintegrations per minute per gram carbon (or dpm/gC), through liquid scintillation counting, preferably according to the Standard Test Method ASTM D6866-16. Said American standard test ASTM D6866 is said to be equivalent to the ISO standard 16620-2. According to said standard ASTM D6866, the testing method may preferably utilize AMS (Accelerator Mass Spectrometry) along with IRMS (Isotope Ratio Mass Spectrometry) techniques to quantify the bio-based content of a given product.

In the present invention, the expression “δ¹³C” refers to the mean isotopic deviation of carbon-13. During photosynthesis, the assimilation of carbonic gas by plants occurs according to 3 principle types of metabolism: metabolism C₃, metabolism C₄ and metabolism CAM. The three photosynthetic processes from C₃, C₄ or CAM plants will generate isotopic effects, in particular the ¹³C isotopic effect, which helps traceability of the botanic origins. Away from industrial activity, atmospheric carbon dioxide displays a mean isotopic deviation of about δ¹³C=−8‰ all over the world. The effect of CO₂ integration by the plant leads to a decrease of ¹³C isotopic ratio in plants of about −20‰ for plants with a C₃ photosynthetic pathway. The C₃ photosynthetic pathway is very discriminative toward ¹³C, whereas C₄ plant discrimination toward ¹³C is lower. As a result, the ¹³C/¹²C isotopic deviation is only lowered by about −3-4‰. As a consequence, δ¹³C isotopic deviation of plants will vary depending of the photosynthetic mechanism. Plants with a photosynthetic metabolism of the C₃ type, such as rice and wheat, display a mean isotopic deviation δ¹³C of about −28‰. Meanwhile, plants with a C₄ photosynthetic mechanism, such as maize, will display a mean isotopic deviation of about δ¹³C=−14‰. These ranges of δ¹³C are typically measured when the plant itself is analysed. Molecules extracted from such plants may have slightly different δ¹³C.

Glyoxylic Acid

Glyoxylic acid is a basic organic chemical raw material. It can be used to make the fragrance of cosmetics, daily chemicals, and food.

Glyoxylic acid (CHO—COOH) contains an aldehyde group and a carboxyl group in its molecule, and is highly reactive. Glyoxylic acid and its derivatives are very important compounds as intermediates for the preparation of various chemicals such as drug modifiers, cosmetics, perfumes and agricultural chemicals.

Various processes have been known as the preparation process of glyoxylic acid. The processes include, method for recovering as a by-product of glyoxal in the nitric acid oxidation process of acetaldehyde, method for oxidizing glyoxal with nitric acid, chlorine or by electro-chemistry, method for the electrochemical reduction of oxalic acid and method for ozonation of maleic acid.

Glyoxylic acid can also be prepared by oxidation, for example catalytic oxidation, of glyoxal which is transformed from mono ethylene glycol (MEG) by air oxidation using silver catalyst in a gas phase reaction at a temperature of 300-700° C.

Glyoxylic acid is very soluble in water and slightly soluble in ethanol, ethyl ether, and benzene.

Glyoxylic acid exists in hydrated form in aqueous solution.

Glyoxylic acid is the source material of the selective hydrogenation of the present invention. The form of the glyoxylic acid is not particularly limited.

Glyoxylic acid is usually provided in solid form, or in an aqueous solution with a concentration of 15-70 wt % industrially, more commonly 40-50 wt %.

Glyoxylic acid may be bio-based glyoxylic acid or non-bio-based glyoxylic acid. According to a preferred embodiment of the present invention, glyoxylic acid has a bio-based carbon content above 50% is hereafter also called “bio-based glyoxylic acid”. Bio-based glyoxylic acid according to the invention may have a bio-based carbon content above 60%, preferably between 75% and 100%, more preferably between 90% and 100%, more preferably between 95% and 100%, more preferably between 98% and 100%, and more preferably between 99% and 100%. Bio-based and non-bio-based glyoxylic acid may be purchased from several producers. Some methods for producing bio-based glyoxylic acid are disclosed in the prior art. In particular, different biochemical processes are available. For instance, U.S. Pat. No. 5,219,745 discloses an industrially advantageous process for biochemical production of glyoxylic acid. Alternatively, bio-based glyoxylic acid may be produced according to well-known industrial methods (see for instance “Glyoxylic Acid” in Ullmann's Encyclopedia of Industrial Chemistry, G. MATTIODA and Y. CHRISTIDIS, Vol. 17 p. 89-92, 2012) starting from bio-based feedstock, like bio-based ethanol or bio-based glycerol.

Because of the bio-sourcing, the raw bio-based glyoxylic acid may contain some impurities. Said impurities may be specific to the origin of the compound.

The bio-based glyoxylic acid used in the present invention may preferably displays a mean isotopic 13C deviation of from −29‰ to −7‰, preferably from −28‰ to −9‰, more preferably from −27‰ to −10‰, most preferably from −28‰ to −25‰.

According to another aspect, the bio-based glyoxylic acid used in the present invention may display a mean isotopic 13C deviation of from −7 to −3‰, preferably from −6‰ to −5‰.

Catalyst

In the present invention, glyoxylic acid is selectively hydrogenated to produce glycolic acid using a catalyst comprising at least one transition metal element (hereinafter also mentioned as hydrogenation metal catalyst, or simply the catalyst).

In one embodiment of the present invention, the hydrogenation metal catalyst to be used is in solid form. In one embodiment of the present invention, the selective hydrogenation reaction is carried out in a liquid medium, or in a liquid form. In one embodiment of the present invention, the hydrogenation metal catalyst to be used according to the present invention is a heterogeneous catalyst.

In chemistry, heterogeneous catalysis is catalysis where the phase of catalysts differs from that of the reactants or products. The process contrasts with homogeneous catalysis where the reactants, products and catalyst exist in the same phase.

Before the present invention, hydrogenation of glyoxylic acid to glycolic acid has only been proposed by using homogeneous catalysts, which have the disadvantages of high cost and/or difficulty separation/recovery from the products. Hydrogenation of glyoxylic acid to glycolic acid over heterogeneous catalysts has not yet been reported.

In one embodiment of the present invention, the hydrogenation metal catalyst to be used according to the present invention is supported or unsupported on a support.

In one embodiment of the present invention, the hydrogenation metal catalyst to be used according to the present invention comprises at least one transition metal element.

In one embodiment of the present invention, the hydrogenation metal catalyst to be used according to the present invention comprises at least one transition metal element selected from groups 3 to 12 of the Periodic Table of Elements excluding lanthanide and actinide series elements.

In one embodiment of the present invention, the hydrogenation metal catalyst to be used according to the present invention comprises at least one transition metal element selected from groups 8 to 11 of the Periodic Table of Elements.

In one embodiment of the present invention, the hydrogenation metal catalyst to be used according to the present invention comprises at least one transition metal element selected from the group consisting of elements such as Pd, Pt, Ru, Ni, Au, Rh, Fe, Cu, Co and Ag.

In one embodiment of the present invention, the hydrogenation metal catalyst to be used according to the present invention comprises at least one transition metal element selected from the group consisting of elements such as Ni, Ru, Pd and Pt.

In one embodiment of the present invention, the hydrogenation metal catalyst to be used according to the present invention comprises at least one transition metal element selected from the group consisting of elements such as Pd and Ni.

As mentioned above, the hydrogenation metal catalyst may be supported or unsupported. If supported on a support, the support may vary widely. In one embodiment of the present invention, the support is porous. The porous support has a specific surface area of greater than or equal to 40 m²/g, lower than or equal to 2000 m²/g.

The support can notably be a porous metal oxide chosen in the group consisting of aluminum oxide (Al₂O₃), silicon dioxide (SiO₂), titanium oxide (TiO₂), zirconium dioxide (ZrO₂), calcium oxide (CaO), magnesium oxide (MgO), lanthanum oxide (La₂O₃), niobium dioxide (NbO₂), cerium oxide (CeO₂), gallium Oxide (Ga₂O₃) and any combination thereof.

The support can also be a zeolite. Zeolites are substances having a crystalline structure and a unique ability to change ions. People skilled in the art can easily understand how to obtain those zeolites by preparation method reported, such as zeolite L described in U.S. Pat. No. 4,503,023 or by commercial purchase, such as ZSM available from ZEOLYST.

In one embodiment of the present invention, examples of suitable supports may be carbon, activated carbons, TiO₂, Al₂O₃, SiO₂, ZrO₂, molecular sieve, or a combination thereof. In one embodiment of the present invention, the transition metal element is supported on a support, which is not a nanotube. If not supported on a support, the catalyst may be Raney Ni. Raney nickel (also called spongy nickel) is a fine-grained solid composed mostly of nickel derived from a nickel-aluminium alloy.

In one embodiment of the present invention, examples of suitable supports are natural supports such as pumice, diatomaceous earth, etc.

If a supported hydrogenation metal catalyst is used, the metal loading, relative to the total weight of the catalyst, is typically from 0.1 to 50 wt. %, preferably above 0.3 wt. %, more preferably above 0.5 wt. %, even more preferably above 1.0 wt. %, and preferably below 30 wt. %, more preferably below 20 wt. %, even more preferably below 15 wt. %, yet even more preferably below 10 wt. %.

In one embodiment of the present invention, the content of the hydrogenation metal catalyst is in the range of 1-20 wt %, preferably 2-15 wt %, more preferably 3-10 wt %, even more preferably 5-10 wt %, relative to the weight of the glyoxylic acid.

Reaction Medium

In one embodiment of the present invention, glyoxylic acid is selectively hydrogenated to produce glycolic acid in the presence of a reaction medium.

In one embodiment of the present invention, the reaction medium is an aqueous solvent.

In one embodiment of the present invention, the reaction medium consists of water.

In one embodiment of the present invention, the reaction medium consists of water, and the reaction medium comes from the water solvent of the aqueous glyoxylic acid.

In one embodiment of the present invention, the reaction medium consists of water, and the reaction medium comes from the water solvent of the aqueous glyoxylic acid and additional water added into the aqueous glyoxylic acid.

In one embodiment of the present invention, the reaction medium comprises water and at least one another solvent selected from the group consisting of ethanol, acetone, acetonitrile, and methanol.

In one embodiment of the present invention, the reaction medium comprises water and another solvent, wherein the weight of the another solvent is in the range of 5% to 50% relative to the weight of water.

In one embodiment of the present invention, the concentration of glyoxylic acid in the reaction medium is in the range of 1-50 wt % relative to the total weight of glyoxylic acid and the reaction medium, preferably, in the range of 5-40 wt %, more preferably in the range of 5-30 wt %, and even more preferably in the range of 8-20 wt %.

In one embodiment of the present invention, glyoxylic acid is itself in liquid form, and is selectively hydrogenated to produce glycolic acid without the presence of a reaction medium.

Selective Hydrogenation

In one embodiment of the present invention, glyoxylic acid is selectively hydrogenated to produce glycolic acid under heat and/or pressure.

In one embodiment of the present invention, the glyoxylic acid is selectively hydrogenated at a temperature from room temperature to 200° C., preferably in the range of 50-150° C., even more preferably in the range of 80-120° C., and still more preferably in the range of 90-110° C.

In one embodiment of the present invention, during the selective hydrogenation, the temperature of the reaction medium is from room temperature to 200° C., preferably in the range of 50-150° C., even more preferably in the range of 80-120° C., and still more preferably in the range of 90-110° C.

The method to heat the glyoxylic acid and/or the reaction medium is not particularly limited, and is well known to a skilled person in the art.

In one embodiment of the present invention, glyoxylic acid is selectively hydrogenated at a pressure in the range of 1 atm to 50 bars, preferably in the range of 5-30bars, more preferably in the range of 10-20bars, even more preferably in the range of 10-15 bars.

In one embodiment of the present invention, the selective hydrogenation is carried out in the presence of a hydrogen gas.

In one embodiment of the present invention, the selective hydrogenation is carried out in a gas atmosphere consisting of hydrogen gas. The hydrogen gas has a pressure in the range of 1 atm to 50 bars, preferably in the range of 5-30bars, more preferably in the range of 10-20bars, even more preferably in the range of 10-15 bars.

In one embodiment of the present invention, the selective hydrogenation is carried out in a gas atmosphere comprising hydrogen gas and at least one other gas, which is not particularly limited as long as it does not have negative effect on the selective hydrogenation, and is preferably selected from the group consisting of N2, CO2, water vapor, and inert gases, preferably selected from the group consisting of He, Ne and Ar. The hydrogen gas has a partial pressure in the range of 1 atm to 50 bars, preferably in the range of 5-30bars, more preferably in the range of 10-20bars, even more preferably in the range of 10-15 bars. In one embodiment of the present invention, the hydrogen gas constitutes a major portion, for example 90 wt % or above, of the gas atmosphere, while the at least one other gas constitutes the rest.

In one embodiment of the present invention, the glyoxylic acid is selectively hydrogenated with a glyoxylic acid conversion rate of no less than 40%, preferably no less than 50%, more preferably no less than 60%, even preferably no less than 70%, still more preferably no less than 80%, particularly preferably no less than 90%, and most preferably 100%. The glyoxylic acid conversion rate is defined as the percentage of the amount of glyoxylic acid consumed during the reaction to the amount of glyoxylic acid introduced.

In one embodiment of the present invention, the glyoxylic acid is selectively hydrogenated with a glycolic acid selection rate of 40% to 90%, such as 50%, 60%, 70%, 80% or 85%. The glycolic acid selection rate is defined as the percentage of the amount of glycolic acid produced to the amount of glyoxylic acid consumed during the reaction.

In one embodiment of the present invention, the glyoxylic acid is selectively hydrogenated in a duration in the range of 2-10 hours, such as 3, 4, 5, 6, 7, 8, or 9 hours, preferably in the range of 2-4 hours, more preferably of about 3 hours.

In one embodiment of the present invention, the glycolic acid produced by the present invention has a bio-based carbon content above 50% and is hereafter also called “bio-based glycolic acid”.

In one embodiment of the present invention, the glycolic acid of the present invention has a bio-based carbon content above 50% and below 100%, preferably 51%-99%, such as 60%, 70%, 80%, 90%, 95%, 96%, 97% or 98%.

In one embodiment of the present invention, the bio-based glycolic acid produced by the present invention may have a bio-based carbon content above 60%, preferably between 75% and 100%, more preferably between 90% and 100%, more preferably between 95% and 100%, more preferably between 98% and 100%, and more preferably between 99% and 100%.

The bio-based glycolic acid of the present invention may preferably display a mean isotopic 13C deviation of from −29‰ to −7‰, preferably from −28‰ to −9‰, more preferably from −27‰ to −10‰, most preferably from −28‰ to −25‰.

According to another embodiment the glycolic acid produced by the present invention preferably displays a mean isotopic 13C deviation of from −7‰ to −3‰, preferably strictly higher than −7‰ to −3‰, preferentially of from −6.5‰ to −5‰.

According to the present invention, both carbon atoms of the glycolic acid produced by the present invention are from a bio-based origin.

In one embodiment of the present invention, glycolic acid is specifically produced by a method comprising the following steps:
(a) providing an aqueous glyoxylic acid;
(b) subjecting the aqueous glyoxylic acid to selective hydrogenation in the presence of hydrogen and a catalyst comprising at least one transition metal element, thereby obtaining glycolic acid; and
(c) collecting the obtained glycolic acid.

In one embodiment of the present invention, in the step (b), an additional amount of water and/or at least one another solvent is also added.

In the step (b), the sequence of introducing the catalyst, the hydrogen, and optionally the additional amount of water and/or at least one another solvent is not limited. In one embodiment of the present invention, the catalyst, the hydrogen, and optionally the additional amount of water and/or at least one another solvent can be added at one time.

In one embodiment of the present invention, the aqueous glyoxylic acid is stirred, by conventional means known to a skilled person in the art, before, during, or after step (b).

As mentioned above, solid glyoxylic acid can be used to carry out the hydrogenation. In one embodiment of the present invention, step (a) can be specifically carried out by dissolving solid glyoxylic acid in the reaction medium, preferably water only, to produce an aqueous glyoxylic acid.

After the selective hydrogenation is finished, glycolic acid is obtained, and then collected, in particular in the above step (c), by way of conventional means known to a skilled person. In one embodiment of the present invention, in the step (c), the obtained product from step (b) is filtered to remove the heterogeneous catalyst, and heated to remove most of the reaction medium to obtain a high-concentration glycolic acid mother liquor, which is then cooled and crystallized to obtain glycolic acid crystal.

In a further embodiment, the present invention relates to a glycolic acid having a bio-based carbon content above 50%. Preferably the glycolic acid of the present invention has a bio-based carbon content above 50%, preferably 51%-99%, such as 60%, 70%, 80%, 90%, 95%, 96%, 97% or 98%. Preferably, the bio-based carbon content of the glycolic acid of the present invention is below 110%, preferably below 105%, even more preferably below 103%. The bio-based carbon content may be preferentially below or equal to 100%, preferentially strictly below 100%.

The glycolic acid of the present invention generally displays a mean isotopic 13C deviation of from −29‰ to −3‰, preferably −29‰ to −7‰, preferably from −28‰ to −9‰, more preferably from −27‰ to −10‰, most preferably from −28‰ to −25‰. According to another embodiment the glycolic acid of the present invention preferably displays a mean isotopic 13C deviation of from −7‰ to −3‰, preferably strictly higher than −7‰ to −3‰, preferentially of from −6.5‰ to −5‰.

According to the present invention, both carbon atoms of the glycolic acid of the present invention are from a bio-based origin.

The invention will now be further described in examples. The examples are given by way of illustration and are not intended to limit the specification or the claims in any manner.

In the following examples, a series of selective hydrogenation reactions, in accordance with formula I below,

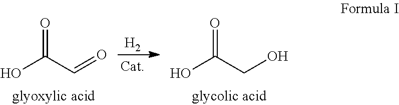

were carried out in a high pressure stainless reactor (PARR4592, 50 ml) with heating equipment. A certain amount of catalyst, glyoxylic acid aqueous solution and additional water if any, as shown in the tables 1-3, were sequentially introduced into the reactor in each of the reactions. The reactor was then filled and purged with H₂ for several times. The H₂ pressure was set at a fixed value, as shown in the tables, and the reactor was heated to a set final temperature for a certain period of time, as also mentioned in the following examples.

HPLC was used to determine the conversion and selectivity of the reactions.

Liquid samples were taken before, during and after the reactions, and analyzed using an Agilent1260 high-pressure liquid chromatography system equipped with a refractive index detector. The compounds were separated in a column (Aminex HPX-87H ion exclusion column) at 303 K, with 0.1 wt % sulfuric acid aqueous solution flowing at 0.45 mL/min as the mobile phase. The retention times and calibration curves of the products were determined by analyzing samples of known concentrations.

Conversion of glyoxylic acid=[(moles of glyoxylic acid introduced−moles of glyoxylic acid remaining in the reaction system at the time of sampling)/(moles of glyoxylic acid introduced)]×100%.

Selectivity of glycolic acid=[(moles of glycolic acid in the reaction system at the time of sampling)/(moles of glyoxylic acid introduced−moles of glyoxylic acid remaining in the reaction system at the time of sampling)]×100%.

### Example 1

As shown in table 1, five commercial available catalysts (all catalysts were obtained from Johnson Matthey, except Raney Ni, which was from Solvay) were used to conduct the selective hydrogenation reaction.

The reactions were carried out under the following conditions: 10 g of 50 wt % glyoxylic acid aqueous solution (available from J&K Scientific), 15 g of additional H₂O, 0.25 g of catalyst, 10 bar continuous H₂ supply and a reaction temperature maintained at 100° C. Each of the reactions were conducted for a duration of 5 hrs.

TABLE 1
hydrogenation of glyoxylic acid to glycolic acid over various
commercial catalysts

| Example | | Glyoxylic | Glycolic |
|---|---|---|---|
| No. | Catalysts | acid conv. (%) | acid Sel. (%) |
| 1.1 | 5% Pd/C | 98 | 61 |
| 1.2 | Raney Ni | 55 | 53 |
| 1.3 | 5% Pt/C | 83 | 65 |
| 1.4 | 5% Ru/C | 21 | 34 |
| 1.5 | 5% Pd/Al₂O₃ | 42 | 34 |

### Example 2

As shown in table 2, 5% Pd/C and Raney Ni were specifically used to conduct the selective hydrogenation reactions. Both the reaction temperature and the H₂ pressure were varied.

The rest reaction conditions were the same as in example 1: 10 g of 50 wt % glyoxylic acid aqueous solution, 15 g of additional H₂O, 0.25 g of catalyst, and a duration of 5 hrs.

TABLE 2
influence of H₂ pressure and reaction temperature on glyoxylic
acid hydrogenation over Pd/C and Raney Ni catalysts.

| Example | | Temp. | H₂ pressure | Conv. | Sel. |
|---|---|---|---|---|---|
| No. | catalysts | (° C.) | (bar) | (%) | (%) |
| 2.1 | 5% Pd/C | 80 | 10 | 56 | 69 |
| 1.1 | 5% Pd/C | 100 | 10 | 98 | 61 |
| 2.2 | 5% Pd/C | 120 | 10 | 100 | 57 |
| 2.3 | 5% Pd/C | 100 | 5 | 65 | 52 |
| 2.4 | 5% Pd/C | 100 | 15 | 100 | 62 |
| 2.5 | Raney Ni | 120 | 10 | 66 | 36 |

### Example 3

As shown in table 3, different glyoxylic acid concentrations were used to conduct the selective hydrogenation reactions. The total weight of the 50 wt % glyoxylic acid aqueous solution and the additional water, if any, was 25 g in each of the reactions. 5 wt % catalyst relative to the actual weight of glyoxylic acid was introduced. The reactions were conducted in the presence of 15 bar H₂, at 100° C., and for 5 hrs.

TABLE 3
influence of glyoxylic acid concentration over
Pd/C and Raney Ni catalysts.

| Example | | Glyoxylic acid | Conv. | Sel. |
|---|---|---|---|---|
| No. | Catalysts | conc. (wt %) * | (%) | (%) |
| 3.1 | 5% Pd/C | 5 | 100 | 83 |
| 3.2 | Raney Ni | 10 | 69 | 69 |
| 3.3 | 5% Pd/C | 10 | 100 | 74 |
| 2.4 | 5% Pd/C | 20 | 100 | 62 |
| 3.4 | 5% Pd/C | 50 | 53 | 33 |

* calculated on basis of the total weight 25 g of the 50 wt % glyoxylic acid aqueous solution and the additional water, if any.

### Example 4

The conversion of glyoxylic acid and the selectivity of glycolic acid at several time points during the reaction in example 1.1 were determined and reported in table 4.

A similar reaction as in example 1.1 was performed in a different reactor: Top Industrie with 130 ml in example 4. The reaction was carried out under the following conditions: 30 g of 50 wt % glyoxylic acid aqueous solution (available from J&K Scientific), 45 g of additional H₂O, 0.75 g of 5% Pd/C, 10 bar continuous H₂ supply and a reaction temperature maintained at 100° C.

The conversion of glyoxylic acid and the selectivity of glycolic acid at several time points during the reaction in example 4 were determined and also reported in table 4.

TABLE 4
Kinetic study of glyoxylic acid hydrogenation over Pd/C using
two different reactors

| | reaction | Conv. | Sel. |
|---|---|---|---|
| Reactor | time (mins) | (%) | (%) |
| PARR, example 1 | 60 | 37 | 43 |
| | 180 | 67 | 52 |
| | 300 | 98 | 61 |
| Top Industrie, example 4 | 15 | 29 | 49 |
| | 30 | 37 | 57 |
| | 60 | 52 | 58 |
| | 180 | 99 | 67 |
| | 300 | 100 | 67 |

### Example 5

A bio-based glyoxylic acid (100% bio-based carbon content, mean 13C deviation of −6.1‰) was reacted under the reaction conditions of Example 1.1 to provide a bio-based glycolic acid.

The bio-based glycolic acid displays a bio-based carbon content of 97% and a mean isotopic deviation of −6.1‰.

1. A method for producing glycolic acid comprising selectively hydrogenating glyoxylic acid, wherein the glycolic acid is selectively hydrogenated in the presence of a catalyst comprising at least one transition metal element.

2. The method according to claim 1, wherein the catalyst is a heterogeneous catalyst.

3. The method according to claim 1, wherein glyoxylic acid is selectively hydrogenated in the presence of a reaction medium.

4. The method according to claim 3, wherein the reaction medium consists of water.

5. The method according to claim 3, wherein the reaction medium comprises water and at least one another solvent selected from the group consisting of ethanol, acetone, acetonitrile, methanol, and combinations thereof.

6. The method according to claim 1, wherein the catalyst is in solid form.

7. The method according to claim 1, wherein the transition metal element is selected from the group consisting of Fe, Co, Ni, Cu, Zn, Ru, Rh, Pd, Ag, Pt and Au.

8. The method according to claim 1, wherein the transition metal element is not supported on a support.

9. The method according to claim 1, wherein the transition metal element is supported on a support selected from the group consisting of activated carbon, Al₂O₃, SiO₂, TiO₂, ZrO₂, molecular sieve, and combinations thereof.

10. The method according to claim 1, wherein the transition metal element is supported on a support, which is not a nanotube.

11. The method according to claim 1, wherein the catalyst is selected from the group consisting of 5 wt % Pd/C, Ni, and combinations thereof.

12. The method according to claim 1, wherein the glyoxylic acid is selectively hydrogenated in the presence of a hydrogen gas.

13. The method according to claim 1, wherein the glyoxylic acid is selectively hydrogenated at a pressure in the range of 1 atm to 50 bars.

14. The method according to claim 1, wherein the glyoxylic acid is selectively hydrogenated at a temperature from room temperature to 200° C.

15. The method according to claim 1, wherein the glyoxylic acid is selectively hydrogenated with a glyoxylic acid conversion rate of no less than 40%.

16. The method according to claim 1, wherein the glyoxylic acid is selectively hydrogenated with a glycolic acid selection rate of 40% to 90%.

17. The method according to claim 1, wherein the concentration of glyoxylic acid in the reaction medium is in the range of 1-50 wt % relative to the total weight of glyoxylic acid and the reaction medium.

18. The method according to claim 1, wherein the content of the catalyst is in the range of 1-20 wt % relative to the weight of the glyoxylic acid.

19. The method according to claim 1, wherein the glyoxylic acid is selectively hydrogenated in a duration in the range of 2-10 hours.

20. A glycolic acid produced by or derived from the method according to claim 1, wherein the glyoxylic acid has a bio-based carbon content of above 50%.

21. The glycolic acid according to claim 20 having a mean isotopic 13C deviation of from −29‰ to −7‰.

22. The glycolic acid according to claim 20 having a mean isotopic deviation of from −7‰ to −3‰.

23. A glycolic acid with a bio-based carbon content of above 50% and below 100%.

24. The glycolic acid according to claim 23 having a mean isotopic 13C deviation of from −29‰ to −3‰.

25. The glycolic acid according to claim 23 having a mean isotopic deviation of from −7‰ to −3‰.

26. (canceled)
